(19) United States
(12) Patent Application Publication
(10) Pub. No.: US 2026/0000615 A1
(43) Pub. Date: Jan. 1, 2026

(54) MULTIPHASE ACTIVE SUBSTANCE PREPARATIONS

(71) Applicant: BASF SE, Ludwigshafen am Rhein (DE)

(72) Inventors: Rene HANSEN, Ballerup (DK); Katrine KORTEGAARD, Ballerup (DK); Inge-Lise KRYLBO, Söborg (DK); Hans-Peter HARZ, Dudenhofen (DE); Wolf PELLETIER, Lampertheim (DE); Stefan BRUHNS, Ludwigshafen am Rhein (DE); Mona Glenstrup JORGENSEN, Ballerup (DK)

(21) Appl. No.: 19/254,704

(22) Filed: Jun. 30, 2025

Related U.S. Application Data
(62) Division of application No. 16/087,200, filed on Sep. 21, 2018.

(30) Foreign Application Priority Data
Mar. 24, 2016 (EP) 16162118.0

(51) Int. Cl. A61K 9/16; A23B 2/729; A23B 2/733; A23K 20/10; A23K 20/163; A23K 50/00; A23L 29/206; A23L 29/219; A23L 29/256; A23L 29/281; A23L 33/15; A23L 33/155; A23P 10/30; A61K 9/10; A61K 9/107; A61K 31/05; A61K 31/07; A61K 31/215; A61K 31/355; A61K 31/365; A61K 31/375; A61K 31/59; A61K 31/593; A61K 45/06

(52) U.S. Cl. CPC A61K 9/1658; A23B 2/729; A23B 2/733; A23K 20/10; A23K 20/163; A23K 50/00; A23L 29/206; A23L 29/219; A23L 29/256; A23L 29/284; A23L 33/15; A23L 33/155; A23P 10/30; A61K 9/107; A61K 9/1617; A61K 9/1664; A61K 31/05; A61K 31/07; A61K 31/215; A61K 31/355; A61K 31/365; A61K 31/375; A61K 31/59; A61K 31/593; A61K 45/06; A23V 2002/00; A23V 2200/02; A23V 2250/211; A23V 2250/50; A23V 2250/502; A23V 2250/5118; A23V 2250/5432; A23V 2250/702; A23V 2250/71; A23V 2250/7106; A61K 9/10; A61K 9/1682

(57) ABSTRACT

The present invention relates to solid, at least three-phase active substance preparations, in which two separate phases are embedded multiparticulately into a coherent, active substance-free and antioxidant-free phase, where one of the embedded phases comprises at least one oxidation-sensitive active substance and the second comprises at least one antioxidant. The invention also relates to a method for producing these active substance preparations, and to the use thereof in food supplements, foods, feeds, body care products, and pharmaceuticals.

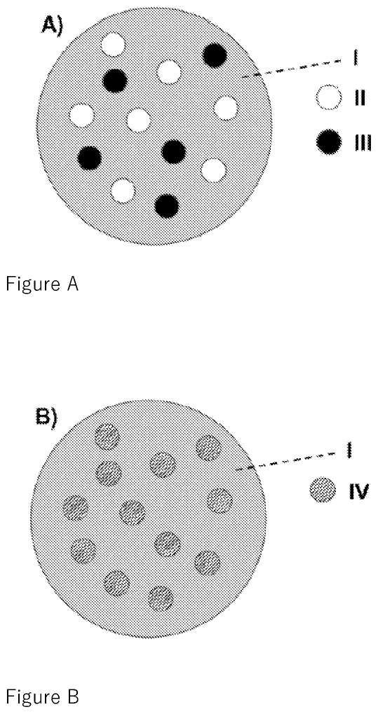

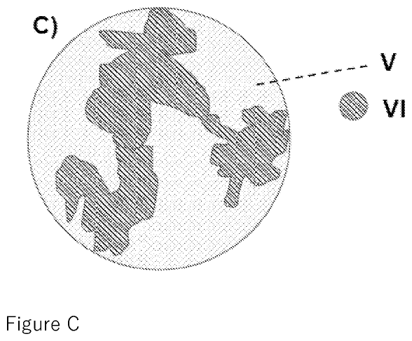

### CROSS-REFERENCE TO RELATED APPLICATIONS

This application is a division of U.S. application Ser. No. 16/087,200, filed Sep. 21, 2018, which is a National stage application (under 35 U.S.C. § 371) of PCT/EP2017/054723, filed Mar. 1, 2017, which claims benefit of European Application No. 16162118.0, filed Mar. 24, 2016, all of which are incorporated herein by reference in their entirety.

### Multiphase Active Substance Preparations

The present invention relates to solid, at least three-phase active compound preparations, in which two separate phases are embedded multiparticulately into a coherent, active compound-free and antioxidant-free phase, where one of the embedded phases comprises at least one oxidation-sensitive active compound and the second comprises at least one antioxidant. The invention also relates to a method for producing these active compound preparations, and to the use thereof in food supplements, foods, feeds, body care products, and pharmaceuticals. In particular, the invention relates to active compound preparations which comprise retinoids and/or vitamin D as embedded, oxidation-sensitive active compound. The active compound preparations of the invention exhibit an improved shelf life by comparison with the prior art.

### BACKGROUND OF THE INVENTION

It is known that certain active compounds are oxidation-sensitive and exhibit stability problems during storage. By oxidation-sensitive, the invention means the capacity of the active compound to react with an oxidizing substance under standard conditions (i.e., between +5° C. and +40° C. at 1013.25 hPa). The oxidation reactions are intended here to include the transfer of oxygen to the active compound, the abstraction of hydrogen, and the giving-up of electrons, optionally with the giving-up of protons. To increase the stability of the active compound toward oxidative degradation, it is advantageous for antioxidants to be added to the active compound preparations. Normally in this case, antioxidants and active compounds are jointly premixed and then added to the remaining constituents.

EP 0065193 discloses a method for producing finely divided carotenoid and/or retinoid products in powder form. It is known therefrom that in the production of active compound preparations, antioxidants may be added either to the aqueous solvent phase or to the active compound-containing solvent phase, but are preferably premixed jointly with the active compound and optionally amphiphilic stabilizers in the solvent phase.

EP 0239086 discloses a method for producing finely divided, water-dispersible carotenoid preparations. From this it is known, for active compound preparations, that the amphiphilic antioxidant ascorbyl palmitate can be mixed into an aqueous protective colloid solution and so assists the formation of a two-phase oil-in-water emulsion, whose oil phase consists of a mixture of carotenoid and antioxidant in carrier oil.

DE 4424085 discloses preparations of fat-soluble active compounds, these preparations being dispersible in cold water. From this it is known, for active compound preparations, that the amphiphilic antioxidant ascorbyl palmitate can be mixed into an aqueous protective colloid solution with partly degraded soy protein and so assists the formation of a two-phase oil-in-water emulsion, whose oil phase comprises oil-soluble and/or fat-soluble active compounds. This oil phase may comprise antioxidants such as butylated hydroxytoluene (BHT) or tocopherol as what are called stabilizers.

EP 0807431 discloses a process for producing carotenoid mixtures. From this is it known, for active compound preparations, that the amphiphilic antioxidant ascorbyl palmitate can be mixed into an aqueous carotenoid suspension, and so assists the formation of a two-phase oil-in-water emulsion, by homogenization of this carotenoid suspension with pressure and heat. Together with the carotenoid, it is also possible to add antioxidants such as dl-α-tocopherol.

EP 1186292 discloses solid preparations having a multicore structure. From this it is known that, following redispersion of at least two different, active compound-containing dry powders, and by reconversion of the mixture into a dry powder, it is possible to prepare an active compound preparation having a multiphase multicore structure. These cores each contain different mixtures of active compounds and optionally antioxidants.

### BRIEF SUMMARY OF THE INVENTION

Surprisingly it has now been found that by adding the antioxidant to the aqueous phase, with subsequent, separate addition of the active compound, a marked increase is achieved in the shelf life of the active compound preparation. The increase in shelf life is achieved both in comparison with the joint addition of the premix of active compound and antioxidant, and with the addition first of the active compound and then, subsequently, of the antioxidant to the aqueous phase.

This surprising finding of the outstanding stabilization is thought to be founded in the development of separate, multiparticulate phases, containing either antioxidant or active compound, which are present in a form in which they are embedded in the coherent phase, characterizing the active compound preparation of the invention. In accordance with the invention, the term “separate” here means spatially separate from one another.

### DETAILED DESCRIPTION OF THE INVENTION

The oxidation-sensitive active compound embedded in the coherent phase is preferably a poorly water-soluble, oxidation-sensitive active compound. “Poorly water-soluble” means in the sense of the invention that the substance has a solubility under standard conditions (temperature 25° C., atmospheric pressure 1013.25 hPa) of less than 0.1 wt % in highly purified water in accordance with the provisions of the European Pharmacopeia Ph.Eur. 8.0. In particular, the first phase embedded into the coherent phase, this first phase comprising a poorly water-soluble, oxidation-sensitive active compound, is antioxidant-free, and the second phase embedded into the coherent phase, that phase comprising an antioxidant, is active compound-free.

Antioxidant-free in the sense of the invention means that the phase comprising a poorly water-soluble, oxidation-sensitive active compound contains on average less than 1000 ppm, preferably less than 100 ppm, and more preferably less than 10 ppm of antioxidant.

Vice versa, active compound-free in the sense of the invention means that the phase comprising an antioxidant contains on average less than 1000 ppm, preferably less than 100 ppm, and more preferably less than 10 ppm of poorly water-soluble, oxidation-sensitive active compound.

Among the poorly water-soluble, oxidation-sensitive active compounds of the invention, retinoids and/or vitamin D are particularly preferred.

Retinoids for the purposes of the present invention are vitamin A alcohol (retinol) and its derivatives such as vitamin A aldehyde (retinal), vitamin A acid (retinic acid), and vitamin A esters (e.g., retinyl acetate, retinyl propionate, retinyl stearate, and retinyl palmitate). The term “retinoids” embraces the all-trans and cis compounds, preferably the all-trans and geometric cis compounds, and more preferably the all-trans compounds.

Vitamin D in accordance with the invention refers to vitamin D₃ and vitamin D₂ and their biologically active metabolites, such as 1-α-hydroxy-vitamin D₃ and 25-hydroxy-vitamin D₃.

Especially preferred here in accordance with the invention are retinyl acetate, retinyl palmitate and/or vitamin D₃.

In the coherent phase, the active compound preparation of the invention comprises at least one protective colloid, which is selected from hydrocolloids of plant and/or animal origin. These hydrocolloids may have been physically and/or chemically modified.

The protective colloids are preferably selected from the group of plant gums, modified plant gums, gelatin, modified gelatin, modified starch, lignosulfonates, chitosans, carrageenans, caseins, caseinates, whey proteins, zeins, modified celluloses, pectins, modified pectins, plant proteins and modified plant proteins, or mixtures thereof.

Plant gums here in accordance with the invention refer to agar, alginic acid, alginate, chicle, dammar, marshmallow extracts, gellan, guar seed meal, gum arabic, gum ghatti, gum from plantain seed husk, gum from spruce sap, gum from larch sap, carob seed flour, karaya, konjac flour, mastic, tara bean gum, tragacanth, or xanthan.

The gelatins include, in accordance with the invention, porcine gelatins, bovine gelatins, fish gelatins, or poultry gelatins, in each case of types A and B and in a Bloom range from 0 to 300.

Plant proteins should be understood in accordance with the invention as pea protein, bean protein, potato protein, lupin protein, soy protein, cereal protein, or peanut protein.

Particularly preferred in the coherent phase, in accordance with the invention, are gelatins, modified starch and/or plant gums.

Especially preferred in accordance with the invention are gelatins, modified starch and/or gum arabic in the coherent phase, and here more particularly active compound preparations which comprise gelatins, modified starch and/or gum arabic in the coherent phase, in combination with the poorly water-soluble, oxidation-sensitive active compounds retinyl acetate, retinyl palmitate and/or vitamin D₃ in the active compound phase.

The active compound preparation of the invention further comprises an antioxidant-containing phase that is embedded in the coherent phase. The at least one antioxidant in this case is selected from the group consisting of tocopherol, butylated hydroxytoluene (BHT), butylated hydroxyanisole (BHA), tert-butylhydroxyquinoline (TBHQ), ethoxyquin (EQ), carnosol, carnosolic acid, or mixtures thereof with amphiphilic antioxidants such as ascorbyl palmitate or ascorbyl stearate. Tocopherol in accordance with the invention refers to dl-α-tocopherol, d-α-tocopherol, β-tocopherol, γ-tocopherol and/or δ-tocopherol.

Of the antioxidants stated, preference is given to those which are poorly water-soluble. “Poorly water-soluble” in the sense of the invention means that under standard conditions (temperature 25° C., atmospheric pressure 1013.25 hPa) they have a solubility of less than 0.1 wt % in highly purified water in accordance with the provisions of the European Pharmacopeia Ph.Eur. 8.0.

With particular preference in accordance with the invention are the poorly water-soluble antioxidants butylated hydroxytoluene (BHT) or tocopherol, and here more particularly active compound preparations which comprise butylated hydroxytoluene (BHT) or tocopherol in the antioxidant-containing phase, in combination with the protective colloids gelatin, modified starch and/or gum arabic in the coherent phase, and in combination with the oxidation-sensitive active compounds retinyl acetate, retinyl palmitate and/or vitamin D₃ in the active compound phase.

The fraction of the poorly water-soluble antioxidants in the active compound preparation is 0.1 to 20 wt %, preferably 0.5 to 15 wt %. Particularly preferred is a fraction of 0.5 to 10 wt % of tocopherol or 2 to 15 wt % of butylated hydroxytoluene (BHT) in the active compound preparation.

The fraction of the oxidation-sensitive active compounds in the active compound preparation is 0.1 to 60 wt %. Where the oxidation-sensitive active compounds comprise vitamin D and/or a retinoid, preference is given to a fraction of 0.1 to 5 wt % of vitamin D and/or 10 to 60 wt % of retinoid, more preferably a fraction of 0.1 to 1 wt % of vitamin D 10 and/or 15 to 40 wt % of retinoid. Vitamin D here is more particularly vitamin D₃, and retinoid more particularly is retinyl palmitate and/or retinyl acetate.

The fraction of the protective colloids in the active compound preparation is 1 to 80 wt %, preferably 10 to 70 wt %. With particular preference the protective colloids here are gelatin, modified starch and/or plant gums.

All data relating to the percentage composition of the active compound preparation of the invention are mass fractions based on the dry mass thereof, with the sum of the percentages of the components making 100%.

In order to improve the production and the applications properties of the active compound preparation, it is useful, optionally, to add further components as auxiliaries and adjuvants. Preferred auxiliaries and adjuvants are plasticizers, emulsifiers, oils, water-soluble salts and/or release agents.

To adapt the mechanical stability of the coherent phase of the active compound preparation, it may be useful to admix the protective colloid with at least one plasticizer, such as polyols, sugars or sugar alcohols, e.g., sucrose, glucose, glucose syrup, starch hydrolyzates, fructose, fructose syrup, lactose, maltose, xylose, arabinose, ribose, trehalose, invert sugars, sorbitol, mannitol, manitol, dextrin, maltodextrin, glycerol, polyether glycols, or isomalt. The isomalt designation stands for a sugar substitute which is also sold under the brand name Palatinit® (Südzucker, Germany). Isomalt is a hydrogenated isomaltulose consisting of approximately equal parts of 6-O-α-D-glucopyranosyl-D-sorbitol and 1-O-α-D-glucopyranosyl-D-mannitol. Plasticizers used with preference are sucrose, glucose syrup, and lactose.

It is possible, moreover, to use emulsifiers to stabilize the phases when producing the active compound preparation of the invention; examples are mono-and diglycerides, monoglycerol fatty acid esters, polyglycerol fatty acid esters, sorbitan fatty acid esters, propylene glycol fatty acid esters, monoglycerol esters of citric acid, sugar esters of fatty acid, or lecithin. Emulsifiers used with preference are mono-and diglycerides, monoglycerol esters of fatty acids, and lecithin.

In certain circumstances it may also be advantageous additionally to use a physiologically approved oil of animal or plant origin, such as, for example, sesame oil, corn germ oil, cottonseed oil, soybean oil, peanut oil, sunflower oil, rapeseed oil, coconut oil, palm oil, olive oil, safflower oil, animal fats, lard, tallow, oils modified by hydrogenation, fractionation or transesterification, or mixtures, in order to dissolve therein at least one poorly water-soluble, oxidation-sensitive active compound and/or a poorly water-soluble antioxidant in the optimum concentration. Preferred oils are corn germ oil, sunflower oil, and rapeseed oil.

Additionally, there may also advantageously be water-soluble inorganic and/or organic salts added to the coherent phase of the active compound preparation, such as, for example, sodium ascorbate, potassium ascorbate, calcium ascorbate, sodium erythorbate, potassium erythorbate, sodium benzoate, potassium benzoate, sodium citrate, potassium citrate, alkali metal phosphates, alkali metal acetates, alkali metal phytates, and mixtures thereof. Preferred salts are sodium ascorbate, sodium benzoate, sodium citrate, and disodium hydrogenphosphate.

In order to prevent unwanted caking and to improve the flowability, it is useful to add poorly water-soluble, finely particulate release agents having an average particle size of less than 15 μm (x50,3 according to DIN ISO 9276-2), which accumulate at the surface of the active compound preparation in powder form. The preferred release agent here is selected from the group consisting of silicon dioxide, hydrophobically modified silica, tricalcium phosphate (TCP), calcium carbonate, sodium carbonate, potassium carbonate, magnesium carbonate, calcium oxide, magnesium oxide, dicalcium diphosphate, calcium silicate, magnesium silicate, magnesium trisilicate, sodium aluminum silicate, talc, kaolin, calcium stearate, magnesium stearate, starches from various botanical sources, cellulose, or mixtures thereof. Particularly preferred here are silicon dioxide, tricalcium phosphate (TCP), hydrophobically modified silica, and corn starch.

The fraction of auxiliaries and adjuvants in accordance with the invention is 0.1 to 70 wt %, preferably 1 to 50 wt %, and more preferably 5 to 30 wt %, based on the dry mass of the active compound preparation of the invention, with the sum of the percentages of the components making 100%.

Another subject of the invention is a method for producing the active compound preparations described at the outset, this method comprising the following steps:
a.) forming an aqueous solution of the components of the coherent phase
b.) mixing the solution from step a.) with at least one poorly water-soluble antioxidant, optionally in solution in an oil, to form a dispersion
c.) mixing the dispersion from step b.) with at least one poorly water-soluble, oxidation-sensitive active compound, optionally in solution in an oil, to form a dispersion and
d.) converting the dispersion from step c.) into a powder product.

The term “dispersion” in the context of the present invention refers both to emulsions and to suspensions, preferably emulsions, and more preferably oil-in-water emulsions.

The dispersions of the invention are produced in apparatus which by input of mechanical energy ensures the fine distribution of the poorly water-soluble components in aqueous solutions. The energy input here is 10⁶ to 10⁹ J/m³, based on the dispersion volume. Production may take place in either a continuous or a batchwise process. Suitable apparatus for these purposes comprises emulsifying vessels equipped with rotor-stator systems, mills such as colloid mills and ball mills, homogenizers such as high-pressure homogenizers, turbine homogenizers, and crown gear dispersing machines, or combinations of these forms of apparatus.

Method steps a.) to c.) for producing the active compound preparations may be performed, in accordance with the invention, either in one apparatus or else in succession in separate apparatus. Preferred is the performance of method steps a.) to c.) in one apparatus, or the performance of method steps a.) and b.) in one apparatus, with a subsequent change of apparatus for the performance of method step c.). Particularly preferred is the performance of method steps a.) to c.) in an emulsifying vessel, or the performance of method steps a.) and b.) in an emulsifying vessel, with subsequent formation of a dispersion, corresponding to method step c.), in a homogenizer.

In embodiments according to the invention, the poorly water-soluble components are in fine distribution in the dispersion. Serving as a measure of the fine distribution is the measurement of the mean particle size (x50,3 according to DIN ISO 9276-2) of the poorly water-soluble components in dispersion by means of laser diffractometry in accordance with ISO 13320-1, using, for example, a Mastersizer 3000 from Malvern Instruments Ltd. Preferred are mean particle sizes of less than 5 μm, more preferably less than 1 μm.

From the dispersion, conversion into a powder product can be accomplished in a conventional way, as for example by granulating, pelletizing, prilling, flaking, compounding, or combined drying and shaping. Among combined drying and shaping, particular preference is given to thin-film drying, spray drying, or modified spray drying with addition of release agent to the drying gas; preferred among the granulating are spray granulation, spray cooling, and modified spray cooling with addition of release agent to the cooling gas. Especially preferred are spray drying, spray cooling, modified spray drying, or modified spray cooling. The powder product thus produced has a mean particle size determined by sieve analysis (x50,3 according to DIN ISO 9276-2) less than 5 mm, preferably less than 1 mm, and more preferably less than 0.5 mm.

For certain applications of the active compound preparations, it may be necessary for the active compound preparation to be dried to a residual moisture content of less than 5 wt % (measured as loss on drying after 3 h in a convection drying cabinet at 105° C.). In the case of certain of the methods described for conversion into a powder product, it may not be possible to achieve this residual moisture content, and in those cases, therefore, there must be subsequent drying of the powder product. This subsequent drying takes place in suitable drying apparatus. Preferred drying apparatus for the subsequent drying comprises contact dryers and convection dryers, with the paddle dryer being particularly preferred among the contact dryers, and the fluidized bed dryer being particularly preferred among the convection dryers. The preferred drying gas is air. The drying temperatures are 40° C. to 200° C.

The invention also relates to active compound preparations obtainable by a method of the invention.

The invention likewise relates to the use of the active compound preparations of the invention in food supplements, foods, feeds, body care products, and pharmaceuticals. For example, the active compound preparations of the invention can be mixed into premixes for multivitamin tablets, baby food, or animal feed. Other examples of use in accordance with the invention are the mixing of the active compound preparations of the invention into water as a basis for liquid products in cosmetology or pharmacy.

Micrographs of the active compound preparations, with contrasting of the respective phases, are shown diagrammatically in the following figures in order to illustrate the invention.

Figure A: inventive active compound preparation as per examples 1-4A with coherent phase (I), embedded into which, separately from one another and in multiparticulate form, are an active compound-containing phase (II) and an antioxidant-containing phase (III).

Figure B: noninventive active compound preparation as per examples 1-4B with coherent phase (I), embedded into which, in multiparticulate form, is a mixed phase (IV) consisting of a joint mixture of active compound and antioxidant.

Figure C: noninventive active compound preparation as per examples 1-4C, with noncontinuously coherent phase (V), embedded into which is an undefined mixed phase (VI) of active compound and antioxidant.

In the text below, the production of the active compound preparations of the invention, the testing of the shelf life, and application are elucidated in more detail by way of example.

### Inventive Example 1A

A gelatin solution was prepared in a heatable emulsifying vessel at 70° C. by bringing 900 g of gelatin (type A, 100 Bloom) into solution in 2500 g of water for 30 minutes by swelling. Added to this gelatin solution were 250 g of a glucose syrup and 70 g of Na₂HPO₄, to give a protective colloid solution. Added to this protective colloid solution were 180 g of BHT, with vigorous stirring* and formation of an emulsion. Then 800 g of melted vitamin A acetate were added with vigorous stirring* emulsified. The emulsion thus prepared was spray-dried via a nozzle in a spraying tower, in which hydrophobic silica (x50,3˜10 μm) was fluidized at 60° C. The particles while still wet were subsequently dried at 60° C. air entry temperature for 30 minutes in the fluidized bed beneath. The active compound preparation thus produced was designated 1A. The mean particle size in dispersion was x50,3˜0.36 μm, the mean particle size of the powder product was x50,3˜230 μm, and the residual moisture content of the powder product was 2.9%.

### Noninventive Example 1B

A gelatin solution was prepared in a heatable emulsifying vessel at 70° C. by bringing 900 g of gelatin (type A, 100 Bloom) into solution in 2500 g of water for 30 minutes by swelling. Added to this gelatin solution were 250 g of a glucose syrup and 70 g of Na₂HPO₄, to give a protective colloid solution. 180 g of BHT and 800 g of melted vitamin A acetate were mixed separately and this mixture was added to the protective colloid solution with stirring. Following the addition, an emulsion was prepared with vigorous stirring *. The emulsion thus prepared was spray-dried via a nozzle in a spraying tower, in which hydrophobic silica (x50,3˜10 μm) was fluidized at 60° C. The particles while still wet were subsequently dried at 60° C. air entry temperature for 30 minutes in the fluidized bed beneath. The active compound preparation thus produced was designated 1B. The mean particle size in dispersion was x50,3˜0.35 μm, the mean particle size of the powder product was x50,3˜240 μm, and the residual moisture content of the powder product was 2.6%.

### Noninventive Example 1C

A gelatin solution was prepared in a heatable emulsifying vessel at 70° C. by bringing 900 g of gelatin (type A, 100 Bloom) into solution in 2500 g of water for 30 minutes by swelling. Added to this gelatin solution were 250 g of a glucose syrup and 70 g of Na₂HPO₄, to give a protective colloid solution. Added to this protective colloid solution were 800 g of melted vitamin A acetate, with vigorous stirring *. Then 180 g of BHT were added and an emulsion was prepared with vigorous stirring *. The emulsion thus prepared was spray-dried via a nozzle in a spraying tower, in which hydrophobic silica (x50,3˜10 μm) was fluidized at 60° C. The particles while still wet were subsequently dried at 60° C. air entry temperature for 30 minutes in the fluidized bed beneath. The active compound preparation thus produced was designated 1C. The mean particle size in dispersion was x50,3˜5.2 μm, the mean particle size of the powder product was x50,3˜220 μm, and the residual moisture content of the powder product was 2.6%.

### Inventive Example 2A

A gum arabic solution was prepared in a heatable emulsifying vessel at 70° C. by bringing 1100 g of gum arabic into solution in 2500 g of water for 30 minutes by swelling. Added to this gum arabic solution were 300 g of sucrose and 20 g of Na ascorbate, to give a protective colloid solution. Added to this protective colloid solution were 70 g of DL-α-tocopherol and 1 g of ascorbyl palmitate, with vigorous stirring* and formation of an emulsion. Then 400 g of vitamin A palmitate were added and emulsified with vigorous stirring*. The emulsion thus prepared was spray-dried via a nozzle in a spraying tower at 120° C. air entry temperature and 90° C. air exit temperature. The particles while still wet were subsequently dried at 60° C. air entry temperature for 30 minutes in the fluidized bed beneath. The active compound preparation thus produced was designated 2A. The mean particle size in dispersion was x50,3˜0.63 μm, the mean particle size of the powder product was x50,3˜140 μm, and the residual moisture content of the powder product was 3.3%.

### Noninventive example 2B

A gum arabic solution was prepared in a heatable emulsifying vessel at 70° C. by bringing 1100 g of gum arabic into solution in 2500 g of water for 30 minutes by swelling. Added to this gum arabic solution were 300 g of sucrose and 20 g of Na ascorbate, to give a protective colloid solution. 70 g of DL-α-tocopherol, 1 g of ascorbyl palmitate, and 400 g of vitamin A palmitate were mixed separately and this mixture was added to the protective colloid solution with stirring. Following the addition, an emulsion was prepared with vigorous stirring *. The emulsion thus prepared was spray-dried via a nozzle in a spraying tower at 120° C. air entry temperature and 90° C. air exit temperature. The particles while still wet were subsequently dried at 60° C. air entry temperature for 30 minutes in the fluidized bed beneath. The active compound preparation thus produced was designated 2B. The mean particle size in dispersion was x50,3˜0.64 μm, the mean particle size of the powder product was x50,3˜180 μm, and the residual moisture content of the powder product was 3.2%.

### Noninventive Example 2C

A gum arabic solution was prepared in a heatable emulsifying vessel at 70° C. by bringing 1100 g of gum arabic into solution in 2500 g of water for 30 minutes by swelling. Added to this gum arabic solution were 300 g of sucrose and 20 g of Na ascorbate, to give a protective colloid solution. Added to this protective colloid solution were 400 g of vitamin A palmitate, with vigorous stirring *. Then 70 g of DL-a-tocopherol and 1 g of ascorbyl palmitate were added and an emulsion was prepared with vigorous stirring *. The emulsion thus prepared was spray-dried via a nozzle in a spraying tower at 120° C. air entry temperature and 90° C. air exit temperature. The particles while still wet were subsequently dried at 60° C. air entry temperature for 30 minutes in the fluidized bed beneath. The active compound preparation thus produced was designated 2C. The mean particle size in dispersion was x50,3˜7.6 μm, the mean particle size of the powder product was x50,3˜170 μm, and the residual moisture content of the powder product was 3.3%.

### Stability Testing for Storage of Active Compound Preparations with Vitamin A in the Premix

The stability of the active compound preparations thus produced was tested in a stress premix test. For this purpose, test specimens of 100 mg in each case of the active compound preparation produced and 4 g of premix mixture were weighed out into 50 ml glass bottles. The premix mixture consisted of 60% of wheat semolina bran, 30% of choline chloride supported at 50% on silica, and 10% of trace element mixture. The trace element mixture consisted of 46% of FeSO₄x7H₂O, 38% of CuSO₄x5H₂O, 12% of ZnO, and 4% of MnO. Following addition of all the ingredients, the test specimens were carefully mixed by hand. These test specimens were stored in a climate chamber at 40° C. and 70% for 4 weeks. The vitamin A content of the test specimens was determined before the beginning of storage and after the end of storage. The vitamin A content was determined in accordance with Regulation (EC) No. 152/2009, Annex IV, Part A. The ratio between the vitamin A contents after and before storage was used to calculate the retention.

The retention values of the examples are compiled in the table below:

| Designation | Retention (%) |
|---|---|
| 1A | 70 |
| 1B | 64 |
| 1C | 62 |
| 2A | 49 |
| 2B | 33 |
| 2C | 29 |

The higher the retention, the better the stability of the active compound preparation in the premix. Comparing the stability of the active compound preparation from the respective inventive examples with the associated noninventive examples (1A with 1B and 1C or 2A with 2B and 2C), the improvement in stability is clearly apparent.

### Inventive Example 3A

A gelatin solution was prepared in a heatable emulsifying vessel at 70° C. by bringing 900 g of gelatin (type A, 240 Bloom) into solution in 2500 g of water by swelling for 30 minutes. Added to this gelatin solution were 250 g of sucrose, to give a protective colloid solution. Added to this protective colloid solution were 60 g of Covi-Ox T 70 EU (mixture of D-α-tocopherol, β-tocopherol, γ-tocopherol, and δ-tocopherol with soybean oil from BASF SE), with vigorous stirring* and formation of an emulsion. Then a premix of 100 g of sunflower oil and 4 g of vitamin D₃ was added and this mixture was emulsified with vigorous stirring *. The emulsion thus prepared was spray-cooled via a nozzle in a spraying tower, in which corn starch (x50,3˜5 μm) was fluidized at 15° C. The particles were subsequently dried while still wet for 30 minutes at an air entry temperature of 60° C. in the fluidized bed beneath. The active compound preparation thus produced was designated 3A. The mean particle size in dispersion was x50,3˜0.34 μm, the mean particle size of the powder product was x50,3˜250 μm, and the residual moisture content of the powder product was 2.7%.

### Noninventive Example 3B

A gelatin solution was prepared in a heatable emulsifying vessel at 70° C. by bringing 900 g of gelatin (type A, 240 Bloom) into solution in 2500 g of water by swelling for 30 minutes. Added to this gelatin solution were 250 g of sucrose, to give a protective colloid solution.

60 g of Covi-Ox T 70 EU (mixture of D-α-tocopherol, β-tocopherol, γ-tocopherol, and δ-tocopherol with soybean oil from BASF SE), 100 g of sunflower oil, and 4 g of vitamin D₃ were mixed separately, and this mixture was added to the protective colloid solution with vigorous stirring. Following the addition, an emulsion was prepared with vigorous stirring*. The emulsion thus prepared was spray-cooled via a nozzle in a spraying tower, in which corn starch (x50,3˜5 μm) was fluidized at 15° C. The particles were subsequently dried while still wet for 30 minutes at an air entry temperature of 60° C. in the fluidized bed beneath. The active compound preparation thus produced was designated 3B. The mean particle size in dispersion was x50,3˜0.37 μm, the mean particle size of the powder product was x50,3˜240 μm, and the residual moisture content of the powder product was 3.2%.

### Noninventive Example 3C

A gelatin solution was prepared in a heatable emulsifying vessel at 70° C. by bringing 900 g of gelatin (type A, 240 Bloom) into solution in 2500 g of water by swelling for 30 minutes. Added to this gelatin solution were 250 g of sucrose, to give a protective colloid solution. Added to this protective colloid solution was a premix of 100 g of sunflower oil and 4 g of vitamin D₃, with vigorous stirring *. Then 60 g of Covi-Ox T 70 EU (mixture of D-α-tocopherol, β-tocopherol, γ-tocopherol, and δ-tocopherol with soybean oil from BASF SE) were added and an emulsion was prepared with vigorous stirring*. The emulsion thus prepared was spray-cooled via a nozzle in a spraying tower, in which corn starch (x50,3˜5 μm) was fluidized at 15° C. The particles were subsequently dried while still wet for 30 minutes at an air entry temperature of 60° C. in the fluidized bed beneath. The active compound preparation thus produced was designated 3C. The mean particle size in dispersion was x50,3˜6.4 μm, the mean particle size of the powder product was x50,3˜280 μm, and the residual moisture content of the powder product was 2.8%.

### Stability Testing for Storage of Active Compound Preparations with Vitamin D₃ in the Premix

The stability of the active compound preparations thus produced was tested in a stress premix test. For this purpose, test specimens of 100 mg in each case of the active compound preparation produced and 4 g of premix mixture were weighed out into 50 ml glass bottles. The premix mixture consisted of 60% of wheat semolina bran, 30% of choline chloride supported at 50% on silica, and 10% of trace element mixture. The trace element mixture consisted of 46% of FeSO₄x7H₂O, 38% of CuSO₄x5H₂O, 12% of ZnO, and 4% of MnO. Following addition of all the ingredients, the test specimens were carefully mixed by hand. These test specimens were stored in a climate chamber at 40° C. and 70% for 4 weeks. The vitamin D₃ content of the test specimens was determined before the beginning of storage and after the end of storage. The vitamin D₃ content was determined according to the methods book of the VDLUFA, Part III, section 13.8.1 (VDLUFA Verlag Darmstadt, 1988). The ratio between the vitamin D₃ contents after and before storage was used to calculate the retention.

The retention values of the examples are compiled in the table below:

| Designation | Retention (%) |
|---|---|
| 3A | 35 |
| 3B | 12 |
| 3C | 17 |

The higher the retention, the better the stability of the active compound preparation in the premix. Comparing the stability of the active compound preparation from the respective inventive example with the associated inventive examples (3A with 3B and 3C), the improvement in stability is clearly apparent.

### Inventive Example 4A

A starch solution was prepared in a heatable emulsifying vessel at 70° C., by bringing 200 g of modified starch (PurityGum 2000 from Ingredion Germany GmbH) into solution in 1500 g of water by swelling for 30 minutes. Added to this starch solution were 1000 g of lactose, 4 g of sodium citrate, 4 g of sodium benzoate, and 20 g of emulsifier (Lamemul K 2000 K from BASF SE), to give a protective colloid solution. Added to this protective colloid solution were 80 g of BHT, with vigorous stirring* and formation of an emulsion. Then 350 g of melted vitamin A acetate were added and the mixture was emulsified on passage through a high-pressure homogenizer**. The emulsion thus prepared was spray-dried via a nozzle in a spraying tower at an air entry temperature of 120° C. and air exit temperature of 90° C. The particles while still wet were subsequently dried at 60° C. in a downstream paddle dryer for 45 minutes, with 5 g of tricalcium phosphate (Phos4Pets 804 from Chemische Fabrik Budenheim KG) being added before the subsequent drying. The active compound preparation thus produced was designated 4A. The mean particle size in dispersion was x50,3˜0.81 μm, the mean particle size of the powder product was x50,3˜160 μm, and the residual moisture content of the powder product was 3.0%.

### Noninventive Example 4B

A starch solution was prepared in a heatable emulsifying vessel at 70° C., by bringing 200 g of modified starch (PurityGum 2000 from Ingredion Germany GmbH) into solution in 1500 g of water by swelling for 30 minutes. Added to this starch solution were 1000 g of lactose, 4 g of sodium citrate, 4 g of sodium benzoate, and 20 g of emulsifier (Lamemul K 2000 K from BASF SE), to give a protective colloid solution. 80 g of BHT and 350 g of melted vitamin A acetate were mixed separately and this mixture was added to the protective colloid solution with vigorous stirring*. After the addition, an emulsion was prepared during passage through a high-pressure homogenizer**. The emulsion thus prepared was spray-dried via a nozzle in a spraying tower at an air entry temperature of 120° C. and air exit temperature of 90° C. The particles while still wet were subsequently dried at 60° C. in a downstream paddle dryer for 45 minutes, with 5 g of tricalcium phosphate (Phos4Pets 804 from Chemische Fabrik Budenheim KG) being added before the subsequent drying. The active compound preparation thus produced was designated 4B. The mean particle size in dispersion was x50,3˜0.68 μm, the mean particle size of the powder product was x50,3˜180 μm, and the residual moisture content of the powder product was 3.4%.

### Noninventive Example 4C

A starch solution was prepared in a heatable emulsifying vessel at 70° C., by bringing 200 g of modified starch (PurityGum 2000 from Ingredion Germany GmbH) into solution in 1500 g of water by swelling for 30 minutes. Added to this starch solution were 1000 g of lactose, 4 g of sodium citrate, 4 g of sodium benzoate, and 20 g of emulsifier (Lamemul K 2000 K from BASF SE), to give a protective colloid solution. Added to this protective colloid solution were 350 g of melted vitamin A acetate, with vigorous stirring*. Then 80 g of BHT were added and an emulsion was prepared during passage through a high-pressure homogenizer**. The emulsion thus prepared was spray-dried via a nozzle in a spraying tower at an air entry temperature of 120° C. and air exit temperature of 90° C. The particles while still wet were subsequently dried at 60° C. in a downstream paddle dryer for 45 minutes, with 5 g of tricalcium phosphate (Phos4Pets 804 from Chemische Fabrik Budenheim KG) being added before the subsequent drying. The active compound preparation thus produced was designated 4C. The mean particle size in dispersion was x50,3˜7.4 μm, the mean particle size of the powder product was x50,3˜170 μm, and the residual moisture content of the powder product was 3.2%.

### Stability Testing for Use of Active Compound Preparations with Vitamin A in Drinking Water

The stability of the active compound preparations thus produced was tested for use in drinking water. For this purpose, test specimens of 100 mg in each case of the active compound preparation produced were weighed out into 10 ml glass bottles and then 8 ml of drinking water were added. These test specimens were stored at 25° C. for 24 hours. The vitamin A content of the test specimens was determined directly at the start of storage (after around 10 minutes) and after the end of storage. The vitamin A content was determined in accordance with Regulation (EC) No. 152/2009, Annex IV, Part A, but without step 5.1. The ratio between the vitamin A contents after and at the start of storage was used to calculate the retention.

The retention values of the examples are compiled in the table below:

| Designation | Retention (%) |
|---|---|
| 4A | 81 |
| 4B | 78 |
| 4C | 54 |

The higher the retention, the better the stability of the active compound preparation on use in drinking water. Comparing the stability of the active compound preparation from the respective inventive examples with the associated noninventive examples (4A with 4B and 4C), the improvement in stability is clearly apparent.
*) Energy input when dispersing (6500 rpm, ULTRA TURRAX UTL 2000 from IKA-Werke GmbH & Co KG) of 3×10⁷ J/m³
**) Energy input when dispersing (350 bar, Microfluidizer M-210 from Microfluidics Corp.) of 2×10⁸ J/m³.

1.-16. (canceled)

17. A method for producing a multiphase active compound preparation, the method comprising the following steps:
a.) forming an aqueous solution of the components of coherent phase comprising a protective colloid
b.) mixing the solution from step a.) with at least one poorly water-soluble antioxidant to form a dispersion
c.) mixing the dispersion from step b.) with at least one poorly water-soluble, oxidation-sensitive active compound to form a dispersion and
d.) converting the dispersion from step c.) into a powder product;
wherein the poorly water-soluble, oxidation-sensitive active compound is selected from the group consisting of retinoids and vitamin D.

18. The method according to claim 17, wherein the protective colloid is selected from the group consisting of plant gums, modified plant gums, gelatin, modified gelatin, modified starch, lignosulfonates, chitosans, carrageenans, caseins, caseinates, whey proteins, zeins, modified celluloses, pectins, modified pectins, plant proteins, modified plant proteins, and mixtures thereof.

19. The method according to claim 17, wherein the at least one poorly water-soluble antioxidant is selected from the group consisting of tocopherol, butylated hydroxytoluene (BHT), butylated hydroxyanisole (BHA), tert-butylhydroxyquinoline (TBHQ), ethoxyquin (EQ), carnosol, carnosolic acid, and mixtures thereof with amphiphilic antioxidants.

20. The method according to claim 17, wherein the oil used in method steps b.) and/or c.) is a physiologically approved oil of animal or plant origin.

21. The method according to claim 17, wherein the at least one poorly water-soluble antioxidant is in solution in an oil.

22. The method according to claim 17, wherein the at least one poorly water-soluble, oxidation sensitive active compound is in solution in an oil.

23. The method according to claim 17, wherein the powder product is a solid, at least three-phase active compound preparation, in which two separate phases are embedded multiparticulately into a coherent, active compound-free and antioxidant-free phase, where one of the embedded phases comprises at least one oxidation-sensitive active compound and the second comprises at least one antioxidant, where active compound-free means on average less than 1000 ppm of active compound and antioxidation-free means on average less than 1000 ppm of antioxidant

24. The method according to claim 23, in which one of the two embedded phases comprises at least one poorly water-soluble, oxidation-sensitive active compound and the second embedded phase comprises at least one poorly water-soluble antioxidant, where poorly water-soluble means a solubility of less than 0.1 wt % in highly purified water under standard conditions. 18 2, in which one of the two embedded phases is antioxidant-free and comprises at least one poorly water-soluble, oxidation-sensitive active compound and the second embedded phase is active compound-free and comprises at least one poorly water-soluble antioxidant.
